(19) United States
(12) Patent Application Publication
(10) Pub. No.: US 2011/0275946 A1
(43) Pub. Date: Nov. 10, 2011

(54) BLOOD PRESSURE MEASUREMENT DEVICE

(75) Inventor: Tameo Ashida, Osaka (JP)

(73) Assignee: OMRON HEALTHCARE CO., LTD., Kyoto (JP)

(21) Appl. No.: 13/185,605

(22) Filed: Jul. 19, 2011

Related U.S. Application Data
(63) Continuation of application No. PCT/JP2009/070579, filed on Dec. 9, 2009.

(30) Foreign Application Priority Data
Jan. 22, 2009 (JP) 2009-012034

(51) Int. Cl. A61B 5/022

(52) U.S. Cl. 600/499

(57) ABSTRACT

A blood pressure measurement device includes a rotation center axis at a position where a distance from an upper arm inserting surface of a living body inserting housing to an elbow rest position center at a “second angle (θ2)=35 degrees”, which is an angle greater than a first angle, is greater than a distance from an upper arm inserting surface of a living body inserting housing to an elbow rest position center at the “first angle (θ1)=20 degrees”. With such a configuration, the blood pressure value can be measured with high accuracy, and the blood pressure measurement device enables the subject to perform the measurement comfortably in a natural position during the measurement.

### TECHNICAL FIELD

The present invention relates to blood pressure measurement devices, and in particular, to a blood pressure measurement device including a mechanism for automatically wrapping an arm band around an upper arm.

### BACKGROUND ART

A blood pressure measurement device including an automatic wrapping mechanism capable of automatically wrapping an arm band around a living body is being widely used in recent years. Japanese Unexamined Patent Publication No. 2006-150143 (Patent Document 1) discloses a blood pressure measurement device mounted with an automatic wrapping mechanism. Advantageously, in such a blood pressure measurement device, stable measurement accuracy is realized because a constant wrapping strength is reproduced for every measurement, and furthermore, a troublesome wrapping operation is not necessary.

The blood pressure measurement device disclosed in Japanese Unexamined Patent Publication No. 2006-150143 (Patent Document 1) includes a main body housing including an elbow rest for placing the elbow when a subject takes the measurement position, and a substantially cylindrical living body inserting housing in which an arm band having a hollow opening, to which the upper arm of the subject is to be inserted, is arranged on an inner peripheral surface. The lower end of the living body inserting housing includes a rotation shaft part coupled to the main body housing, so that the living body inserting housing is rotatably arranged with respect to the main body housing.

In the blood pressure measurement device having the above configuration, when the blood pressure measurement device is mounted on a mount surface of a desk or the like and the subject measures the blood pressure while sitting on a chair, the rotation angle of the living body inserting housing (angle formed by the mount surface and the center axis of the arm band) becomes small for a short subject and the rotation angle of the living body inserting housing becomes large for a tall subject.

The length of the upper arm of a short subject is physically short compared to the length of the upper arm of a tall subject, but the distance from the upper arm inserting surface of the living body inserting housing to the position of the elbow rest becomes disadvantageously long for the short subject as a result of the rotation angle of the living body inserting housing becoming small.

Patent Document 1: Japanese Unexamined Patent Publication No. 2006-150143

Accordingly, the distance from the upper arm inserting surface of the living body inserting housing to the position of the elbow rest becomes long when the rotation angle of the living body inserting housing becomes small in the blood pressure measurement device having a configuration in which the living body inserting housing is rotatably arranged with respect to the main body housing.

Therefore, one or more embodiments of the present invention provides a blood pressure measurement device capable of measuring a blood pressure value with high accuracy and enabling the subject to carry out the measurement comfortably in a natural position during the measurement.

### SUMMARY OF INVENTION

A blood pressure measurement device according to one or more embodiments of the present invention includes a substantially cylindrical living body inserting housing in which an arm band with a hollowing opening, to which an upper arm of a subject is inserted from an axial direction, is arranged on an inner peripheral surface; and a main body housing including an elbow rest for placing an elbow of the subject when the upper arm of the subject is passed through the living body inserting housing to take a measurement position.

The living body inserting housing includes a rotation center axis for an angle formed by a plane including an elbow rest position center in the elbow rest and an arm band center axis of the arm band to be movable in a range between a first angle and a second angle greater than the first angle.

When viewed from the axial direction, the rotation center axis is arranged at a position where a distance from an upper arm inserting surface of the living body inserting housing to the elbow rest position center in the second angle becomes greater than a distance from the upper arm inserting surface of the living body inserting housing to the elbow rest position center in the first angle.

In the blood pressure measurement device according to one or more embodiments of the present invention, the rotation center axis is arranged at a position where the distance from the upper arm inserting surface of the living body inserting housing to the elbow rest position center in the second angle, which is an angle greater than the first angle, becomes greater than a distance from the upper arm inserting surface of the living body inserting housing to the elbow rest position center in the first angle.

Therefore, the distance from the upper arm inserting surface of the living body inserting housing to the elbow rest position becomes short when the rotation angle of the living body inserting housing becomes small, so that even a short subject can perform the measurement comfortably in a natural position during the measurement.

### BRIEF DESCRIPTION OF THE DRAWINGS

FIG. 1 is a plan view of a blood pressure measurement device according to one or more embodiments of the present invention.

FIG. 2 is a front view of the blood pressure measurement device according to one or more embodiments of the present invention.

FIG. 3 is a right side view of the blood pressure measurement device according to one or more embodiments of the present invention.

FIG. 4 is a view showing a state in which a living body inserting housing adopted in the blood pressure measurement device according to one or more embodiments of the present invention is turned.

FIG. 5 is a diagram showing the function blocks of the blood pressure measurement device according to one or more embodiments of the present invention.

FIG. 6 is a view showing a measurement position of a subject according to one or more embodiments of the present invention.

FIG. 7 is a schematic view showing a positional relationship of the living body inserting housing and the elbow rest when viewed from the axial direction of the rotation center axis of the blood pressure measurement device according to one or more embodiments of the present invention.

FIG. 8 is a view showing the position of the rotation center axis of the blood pressure measurement device according to one or more embodiments of the present invention.

FIG. 9 is a 1st view showing the rotation state of the living body inserting housing at the position of the selected rotation center axis P11 in the blood pressure measurement device according to one or more embodiments of the present invention.

FIG. 10 is a 2nd view showing the rotation state of the living body inserting housing at the position of the selected rotation center axis P12 in the blood pressure measurement device according to one or more embodiments of the present invention.

FIG. 11 is a 3rd view showing the rotation state of the living body inserting housing at the position of the selected rotation center axis P13 in the blood pressure measurement device according to one or more embodiments of the present invention.

FIG. 12 is a 4th view showing the rotation state of the living body inserting housing at the position of the selected rotation center axis P14 in the blood pressure measurement device according to one or more embodiments of the present invention.

FIG. 13 is a 5th view showing the rotation state of the living body inserting housing at the position of the selected rotation center axis P15 in the blood pressure measurement device according to one or more embodiments of the present invention.

FIG. 14 is a 6th view showing the rotation state of the living body inserting housing at the position of the selected rotation center axis P16 in the blood pressure measurement device according to one or more embodiments of the present invention.

FIG. 15 is a 7th view showing the rotation state of the living body inserting housing at the position of the selected rotation center axis P17 in the blood pressure measurement device according to one or more embodiments of the present invention.

FIG. 16 is an 8th view showing the rotation state of the living body inserting housing at the position of the selected rotation center axis P21 in the blood pressure measurement device according to one or more embodiments of the present invention.

FIG. 17 is a 9th view showing the rotation state of the living body inserting housing at the position of the selected rotation center axis P22 in the blood pressure measurement device according to one or more embodiments of the present invention.

FIG. 18 is a 10th view showing the rotation state of the living body inserting housing at the position of the selected rotation center axis P23 in the blood pressure measurement device according to one or more embodiments of the present invention.

FIG. 19 is an 11th view showing the rotation state of the living body inserting housing at the position of the selected rotation center axis P24 in the blood pressure measurement device according to one or more embodiments of the present invention.

FIG. 20 is a 12th view showing the rotation state of the living body inserting housing at the position of the selected rotation center axis P25 in the blood pressure measurement device according to one or more embodiments of the present invention.

FIG. 21 is a 13th view showing the rotation state of the living body inserting housing at the position of the selected rotation center axis P26 in the blood pressure measurement device according to one or more embodiments of the present invention.

FIG. 22 is a 14th view showing the rotation state of the living body inserting housing at the position of the selected rotation center axis P27 in the blood pressure measurement device according to one or more embodiments of the present invention.

FIG. 23 is a 15th view showing the rotation state of the living body inserting housing at the position of the selected rotation center axis P31 in the blood pressure measurement device according to one or more embodiments of the present invention.

FIG. 24 is a 16th view showing the rotation state of the living body inserting housing at the position of the selected rotation center axis P32 in the blood pressure measurement device according to one or more embodiments of the present invention.

FIG. 25 is a 17th view showing the rotation state of the living body inserting housing at the position of the selected rotation center axis P33 in the blood pressure measurement device according to one or more embodiments of the present invention.

FIG. 26 is an 18th view showing the rotation state of the living body inserting housing at the position of the selected rotation center axis P34 in the blood pressure measurement device according to one or more embodiments of the present invention.

FIG. 27 is a 19th view showing the rotation state of the living body inserting housing at the position of the selected rotation center axis P35 in the blood pressure measurement device according to one or more embodiments of the present invention.

FIG. 28 is a 20th view showing the rotation state of the living body inserting housing at the position of the selected rotation center axis P36 in the blood pressure measurement device according to one or more embodiments of the present invention.

FIG. 29 is a 21st view showing the rotation state of the living body inserting housing at the position of the selected rotation center axis P37 in the blood pressure measurement device according to one or more embodiments of the present invention.

FIG. 30 is a 22nd view showing the rotation state of the living body inserting housing at the position of the selected rotation center axis P41 in the blood pressure measurement device according to one or more embodiments of the present invention.

FIG. 31 is a 23rd view showing the rotation state of the living body inserting housing at the position of the selected rotation center axis P42 in the blood pressure measurement device according to one or more embodiments of the present invention.

FIG. 32 is a 24th view showing the rotation state of the living body inserting housing at the position of the selected rotation center axis P43 in the blood pressure measurement device according to one or more embodiments of the present invention.

FIG. 33 is a 25th view showing the rotation state of the living body inserting housing at the position of the selected rotation center axis P44 in the blood pressure measurement device according to one or more embodiments of the present invention.

FIG. 34 is a 26th view showing the rotation state of the living body inserting housing at the position of the selected rotation center axis P45 in the blood pressure measurement device according to one or more embodiments of the present invention.

FIG. 35 is a 27th view showing the rotation state of the living body inserting housing at the position of the selected rotation center axis P46 in the blood pressure measurement device according to one or more embodiments of the present invention.

FIG. 36 is a 28th view showing the rotation state of the living body inserting housing at the position of the selected rotation center axis P47 in the blood pressure measurement device according to one or more embodiments of the present invention.

FIG. 37 is a 29th view showing the rotation state of the living body inserting housing at the position of the selected rotation center axis P51 in the blood pressure measurement device according to one or more embodiments of the present invention.

FIG. 38 is a 30th view showing the rotation state of the living body inserting housing at the position of the selected rotation center axis P52 in the blood pressure measurement device according to one or more embodiments of the present invention.

FIG. 39 is a 31st view showing the rotation state of the living body inserting housing at the position of the selected rotation center axis P53 in the blood pressure measurement device according to one or more embodiments of the present invention.

FIG. 40 is a 32nd view showing the rotation state of the living body inserting housing at the position of the selected rotation center axis P54 in the blood pressure measurement device according to one or more embodiments of the present invention.

FIG. 41 is a 33rd view showing the rotation state of the living body inserting housing at the position of the selected rotation center axis P55 in the blood pressure measurement device according to one or more embodiments of the present invention.

FIG. 42 is a 34th view showing the rotation state of the living body inserting housing at the position of the selected rotation center axis P56 in the blood pressure measurement device according to one or more embodiments of the present invention.

FIG. 43 is a 35th view showing the rotation state of the living body inserting housing at the position of the selected rotation center axis P57 in the blood pressure measurement device according to one or more embodiments of the present invention.

FIG. 44 is a first view showing the results obtained from the states of FIG. 9 to FIG. 43.

FIG. 45 is a second view showing the results obtained from the states of FIG. 9 to FIG. 43.

### DETAILED DESCRIPTION OF INVENTION

Hereinafter, embodiments of the present invention will be described in detail with reference to the drawings. A blood pressure measurement device according to one or more embodiments of the present invention detects an arterial pressure pulse wave by compressing an upper arm of a subject, and measures a blood pressure value. The blood pressure measurement device according to one or more embodiments of the present invention includes an automatic arm band wrapping mechanism, which wraps the arm band around the upper arm.

### (Outer Appearance Structure of Blood Pressure Measurement Device 100A)

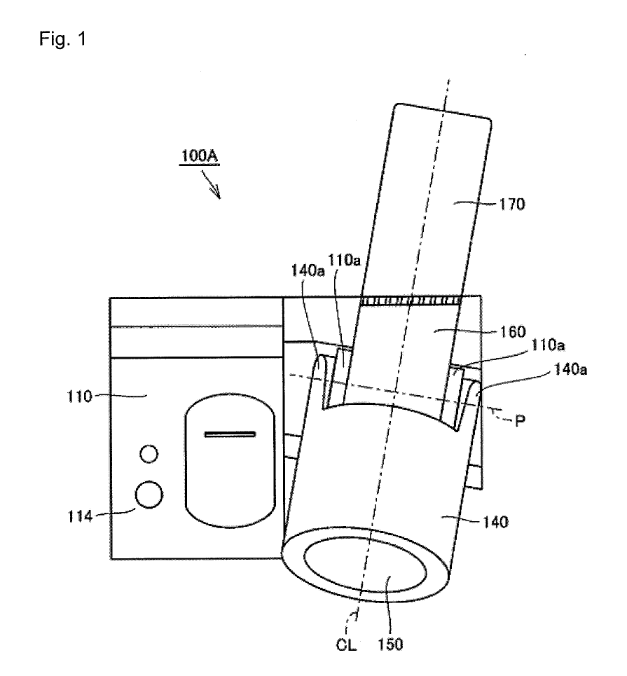

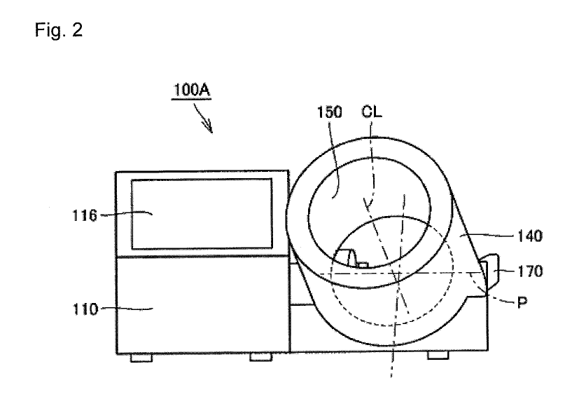

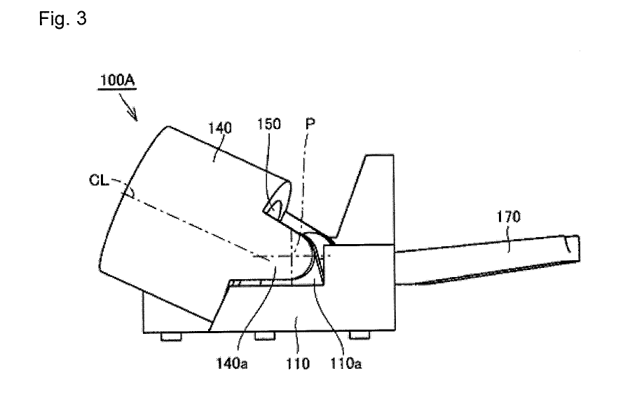

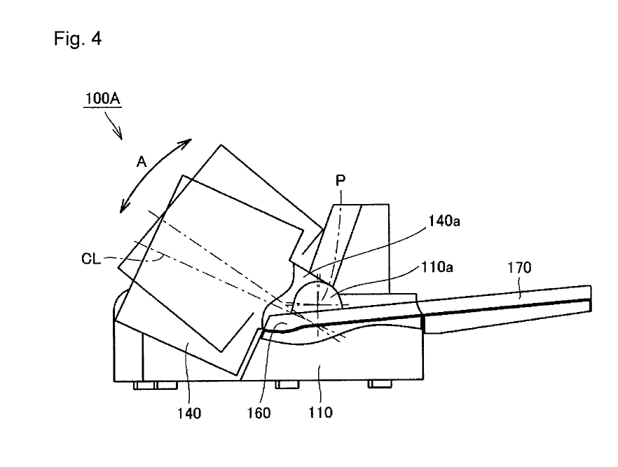

FIG. 1 to FIG. 4 are views each describing an outer appearance structure of a blood pressure measurement device 100A according to one or more embodiments of the present invention, where FIG. 1 is a plan view of a blood pressure measurement device according to one or more embodiments of the present invention, FIG. 2 is a front view of the blood pressure measurement device according to one or more embodiments of the present invention, FIG. 3 is a right side view of the blood pressure measurement device according to one or more embodiments of the present invention, and FIG. 4 is a view showing a state in which a living body inserting housing adopted in the blood pressure measurement device according to one or more embodiments of the present invention is turned.

As shown in FIG. 1 to FIG. 3, the blood pressure measurement device 100A according to one or more embodiments of the present invention includes a substantially cylindrical living body inserting housing 140 to be mounted on a mount surface of a desk and the like and arranged with an arm band 150, which has a hollow opening to which the upper arm of the subject is inserted from an axial direction, on the inner peripheral surface, and a main body housing 110 including an elbow rest 160 for placing the elbow of the subject when the subject passes the upper arm through the living body inserting housing 140 and takes the measurement position. The blood pressure measurement device 100A according to one or more embodiments of the present invention also includes an arm rest 170 for placing the arm of the subject.

An operation unit 114, including various buttons such as a power supply button used to turn ON the power supply, a measurement button for starting the measurement operation, and a display unit operation button for performing the operation of a display unit, is arranged on the upper surface of the main body housing 110. A display unit 116 for displaying the measurement result, the operation guide, and the like is arranged at another position on the upper surface of the main body housing 110.

As shown in FIG. 4, the living body inserting housing 140 is coupled in a freely rotatable manner (direction of arrow A in FIG. 4) with respect to the main body housing 110 by a rotation coupling mechanism including a rotation center axis P. As a specific configuration, a pair of opposing supporting plates 140a, 140a is arranged to project out toward the main body housing 110 side of the living body inserting housing 140, which supporting plates 140a, 140a are turnably coupled to base plates 110a, 110a arranged on the main body housing 110. The position of the rotation center axis P will be described later.

### (Function Blocks of Blood Pressure Measurement Device 100A)

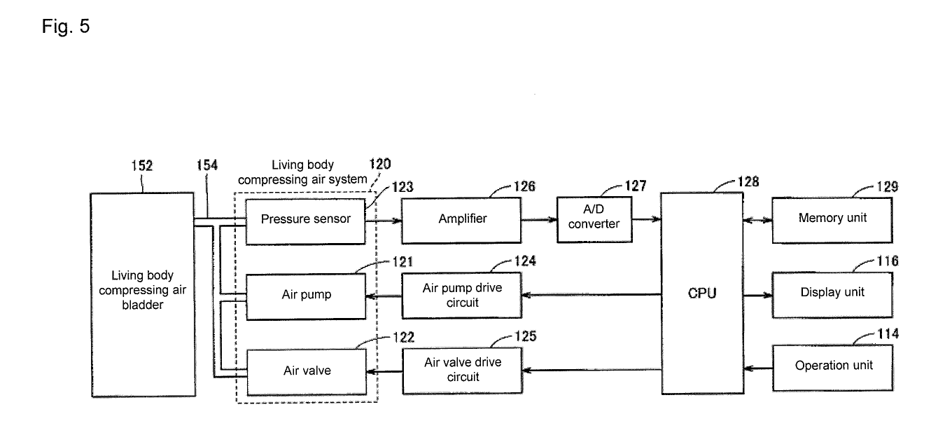

FIG. 5 is a diagram showing the function blocks of the blood pressure measurement device 100A shown in FIG. 1 to FIG. 3. As shown in FIG. 5, a living body compressing air bladder 152 arranged in the arm band is connected to a living body compressing air system 120 by an air tube 154. The operation of the living body compressing air system 120 is controlled by a CPU 128.

The living body compressing air system 120 includes an air pump 121, an air valve 122, and a pressure sensor 123. The air pump 121 is means for pressurizing the lumen of the living body compressing air bladder 152, and is driven by an air pump drive circuit 124 receiving a command from the CPU 128 to send compressed air to the lumen such that the pressure of the lumen of the living body compressing air bladder 152 becomes a predetermined pressure at the time of the measurement.

The air valve 122 is means for maintaining or depressurizing the pressure of the lumen of the living body compressing air bladder 152, which open and close state is controlled by an air valve drive circuit 125 receiving a command from the CPU 128 to maintain or depressurize the pressure of the lumen of the living body compressing air bladder 152, which became a high pressure state by the air pump 121 at the time of the measurement, and to return the lumen of the living body compressing air bladder 152 to atmospheric pressure after the end of the measurement.

The pressure sensor 123 is means for detecting the pressure of the lumen of the living body compressing air bladder 152, and detects the pressure of the lumen of the living body compressing air bladder 152 that changes every second at the time of the measurement and outputs a signal corresponding to the detection value to an amplifier 126. The amplifier 126 amplifies the signal output from the pressure sensor 123 and outputs the same to an ND converter 127. The ND converter 127 digitalizes the analog signal output from the amplifier 126 and outputs the same to the CPU 128.

The CPU 128 performs the control of the living body compressing air system 120 based on the command input to the operation unit 114 arranged in the main body housing 110 of the blood pressure measurement device and outputs the measurement result to the display unit 116 and the memory unit 129. The memory unit 129 is means for storing measurement results.

In the blood pressure measurement device 100A according to one or more embodiments of the present invention, all the function blocks excluding the living body compressing air bladder 152 and the pressure sensor 123 of each function block shown in FIG. 5 are arranged in the main body housing 110, and accommodated in the main body housing 110. The living body compressing air bladder 152 and the pressure sensor 123 are arranged in the living body inserting housing 140.

The living body compressing air bladder 152, and the air pump 121 and the air valve 122 are connected by a flexible air tube, and the pressure sensor 123 and the amplifier 126 are connected by a flexible signal line. As the components accommodated in the main body housing and the components accommodated in the living body inserting housing 140 are connected using the flexible air tube and the signal line, the injection and discharge of air or transmission and reception of signal are performed while following the rotational movement of the living body inserting housing 140.

### (Measurement Position of Subject)

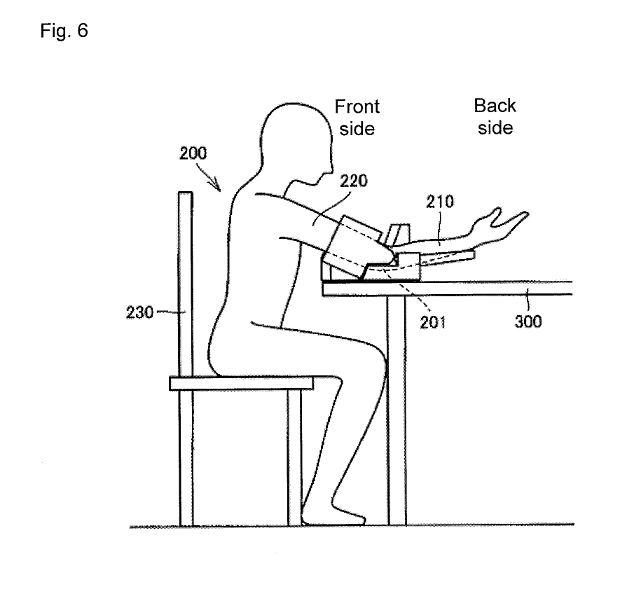

FIG. 6 is a view showing the measurement position of the subject 200 using the blood pressure measurement device 100A according to one or more embodiments of the present invention. The blood pressure measurement device 100A is mounted on the mount surface of the desk 300, and the subject 200 is sitting on a chair 230. The upper arm 220 of the subject 200 is inserted from the axial direction of the living body inserting housing 140. The elbow 201 of the subject 200 is placed on the elbow rest 160, and the arm 210 of the subject 200 is placed on the arm rest 170.

### (Position of Rotation Center Axis P)

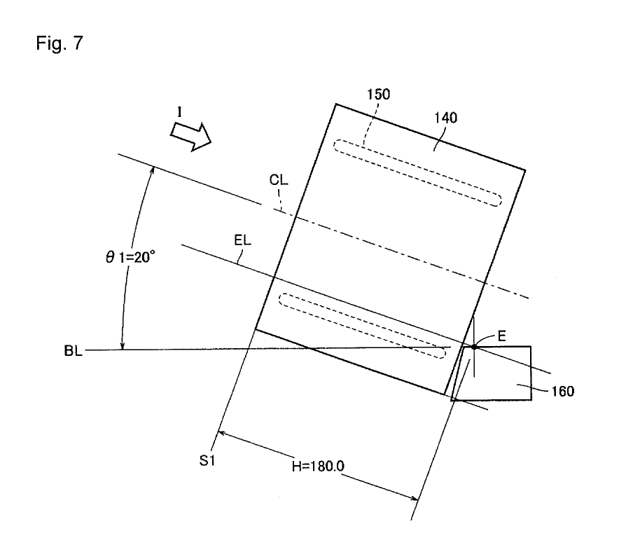

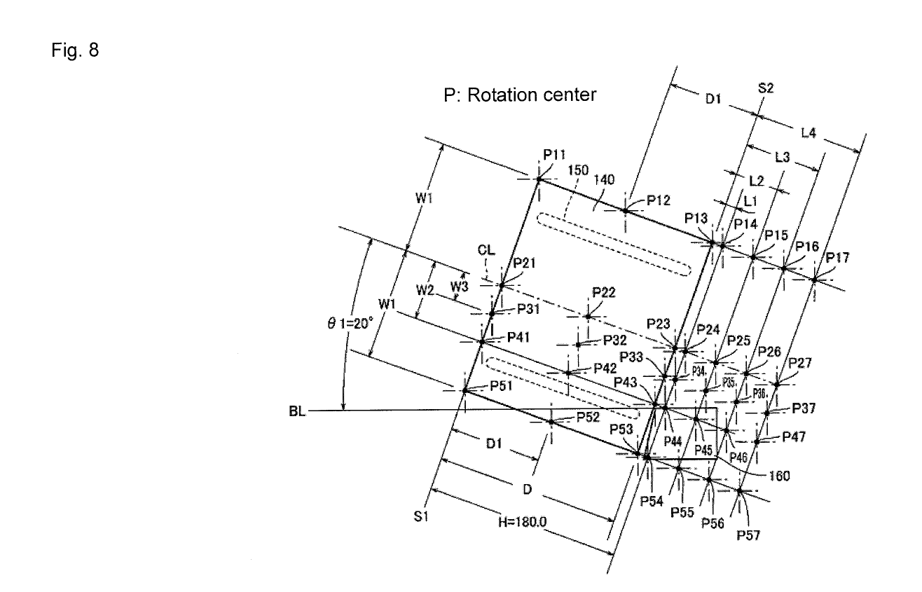

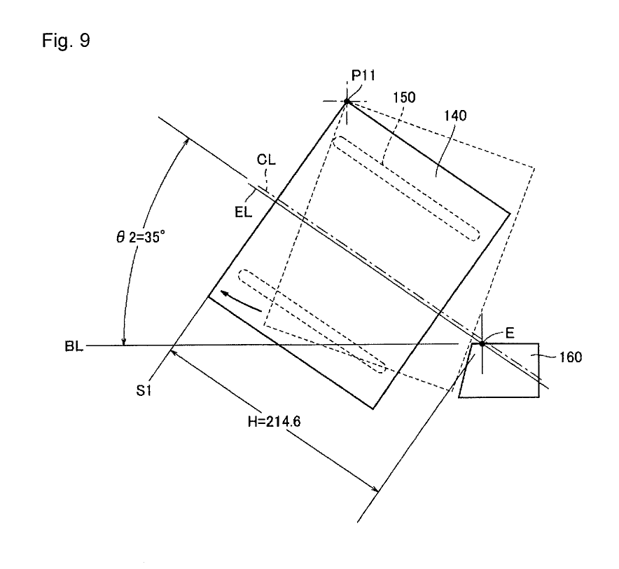

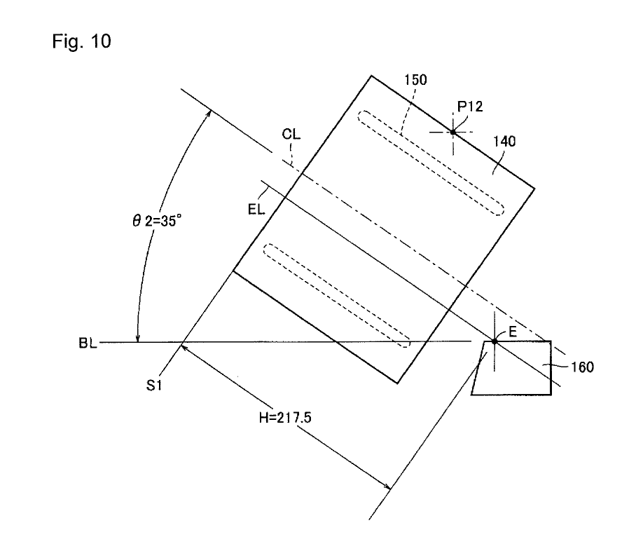

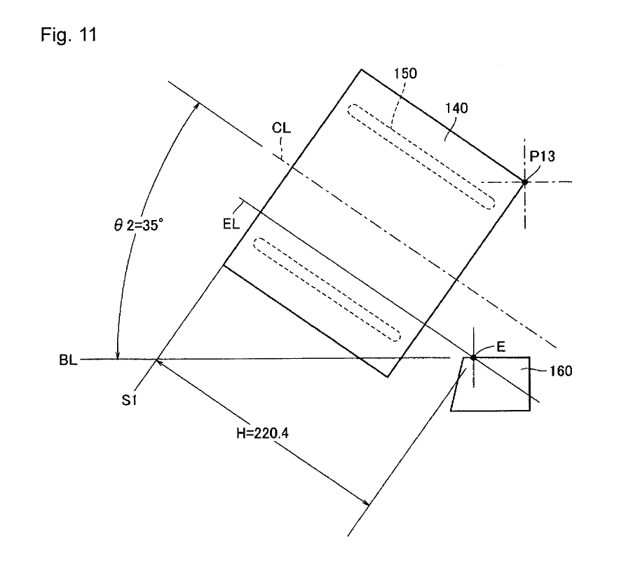

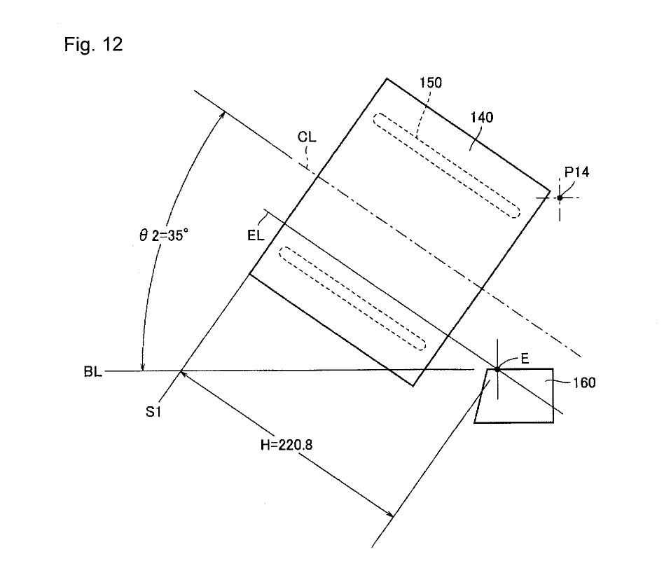

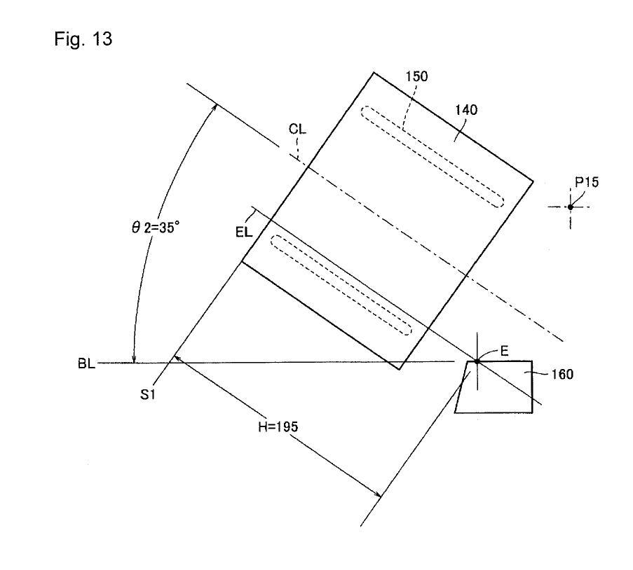

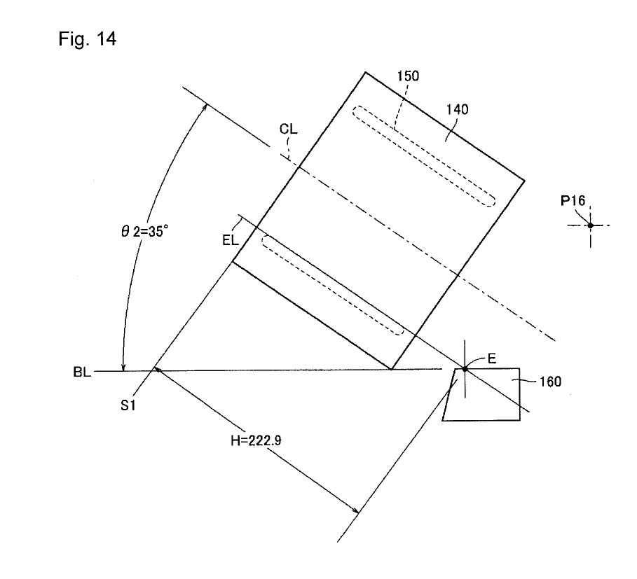

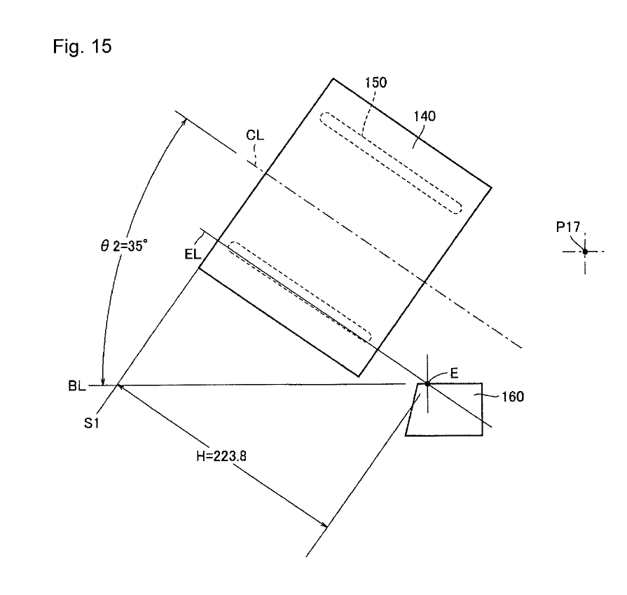

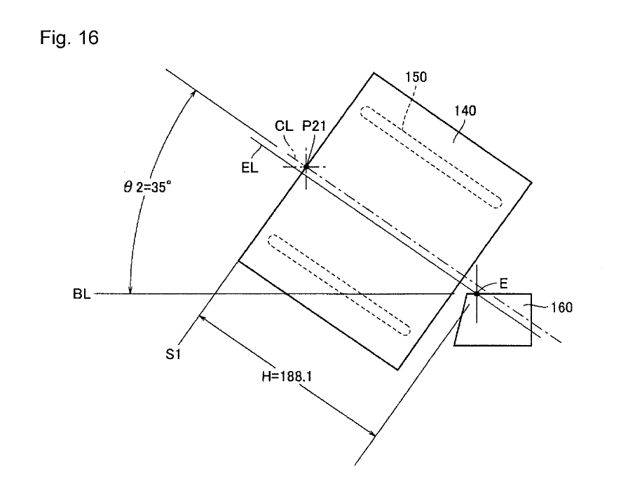

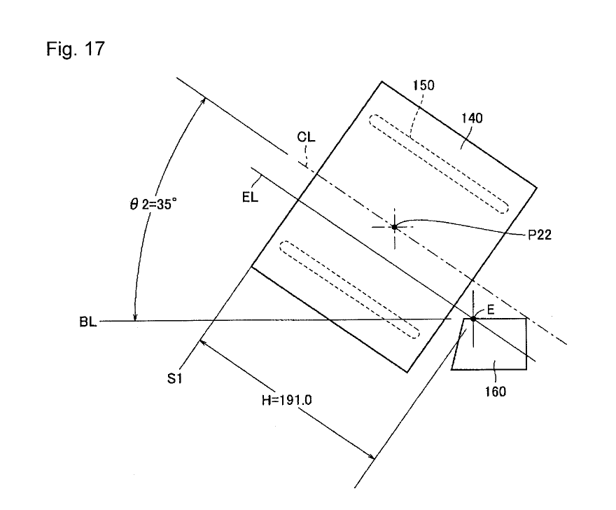

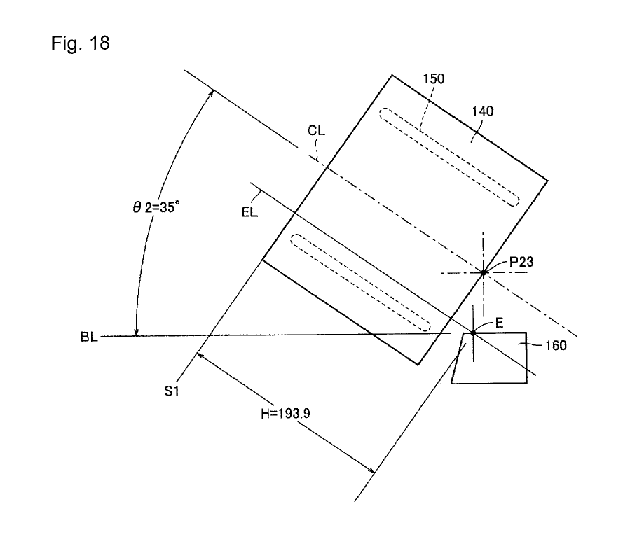

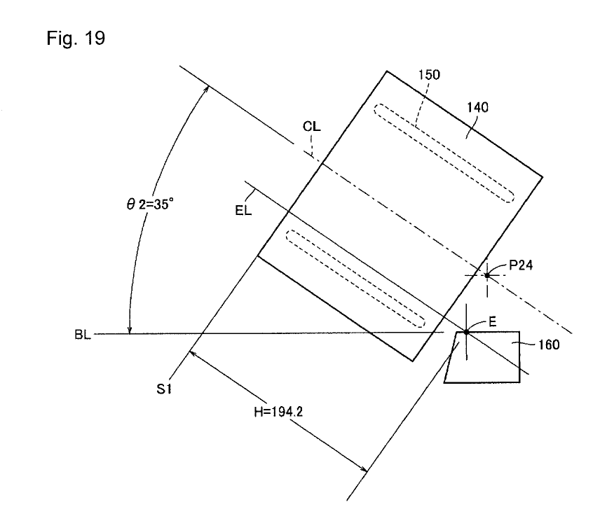

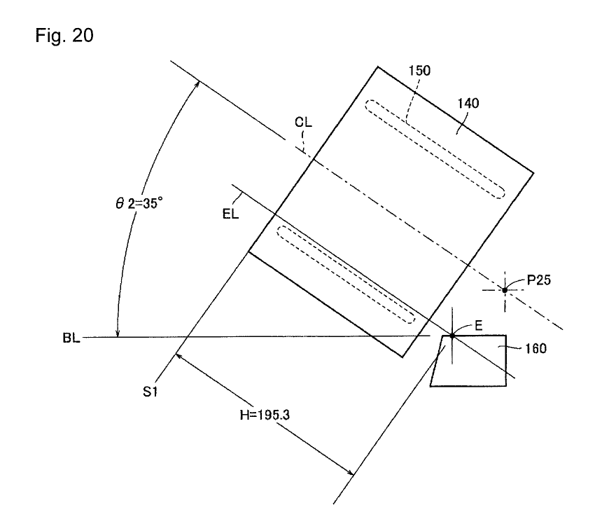

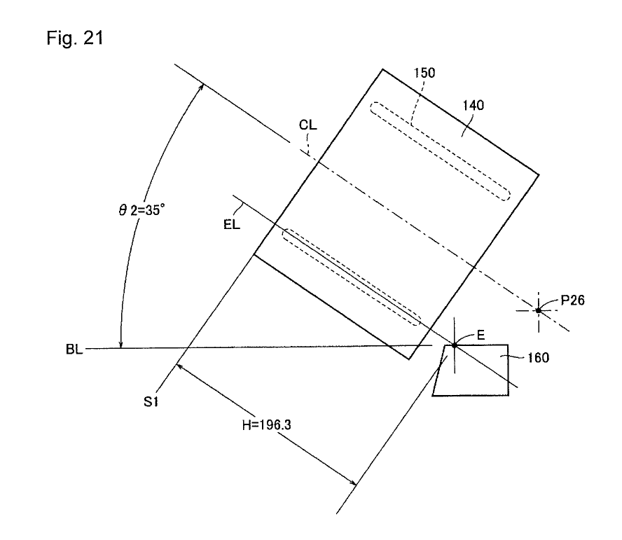

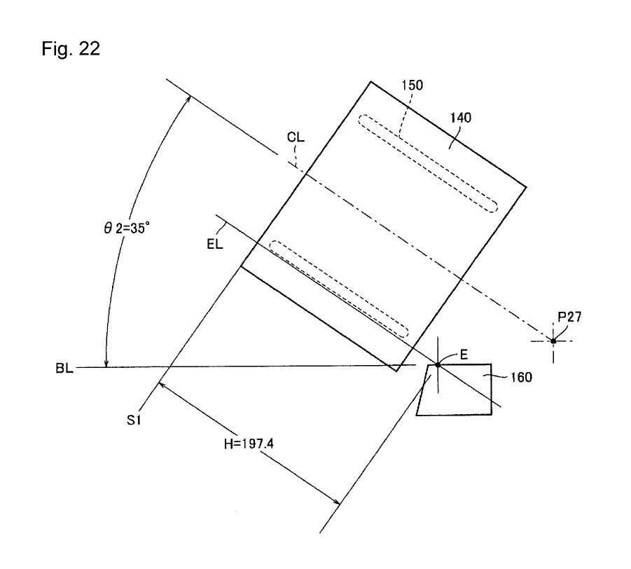

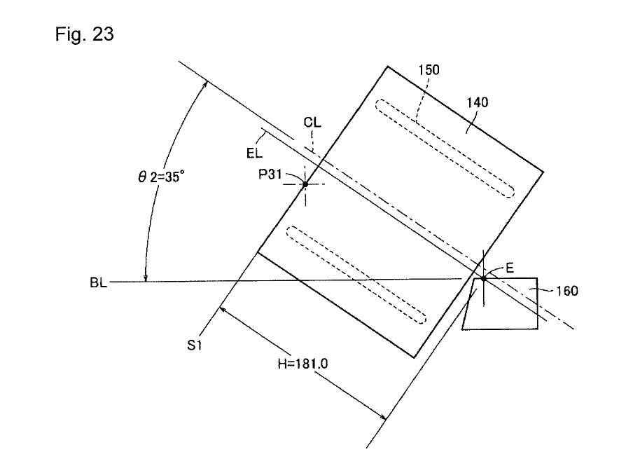

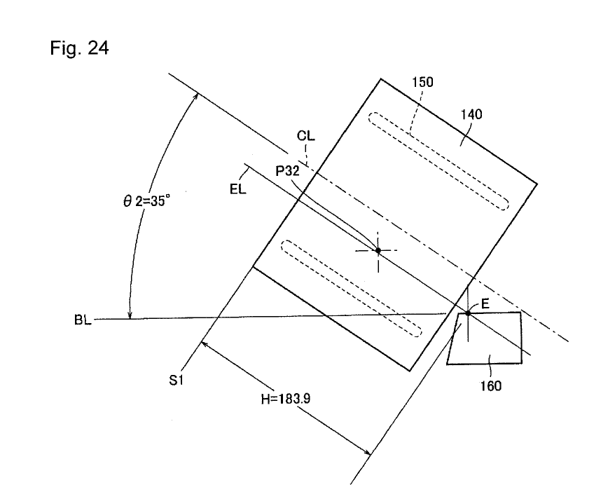

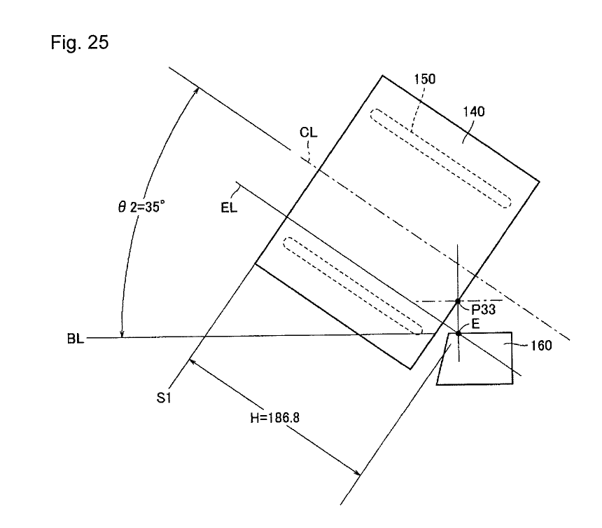

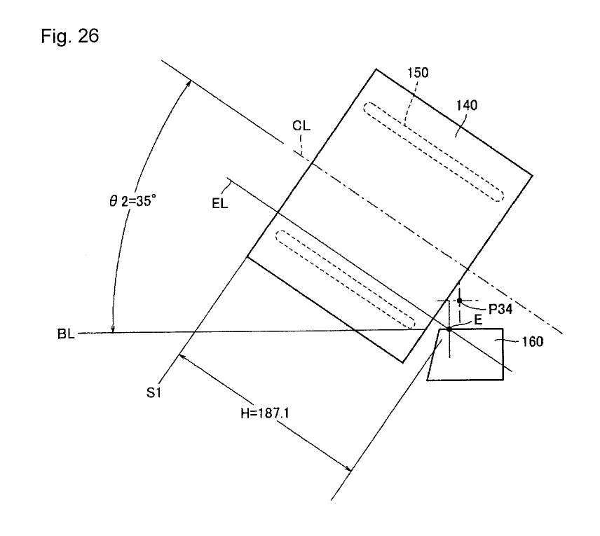

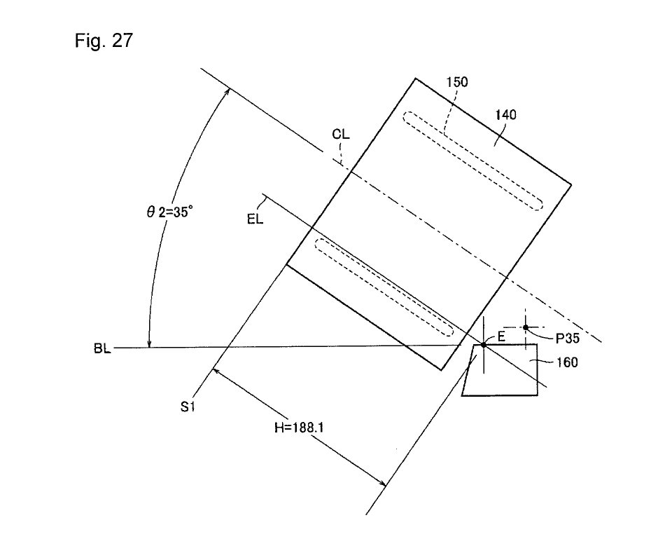

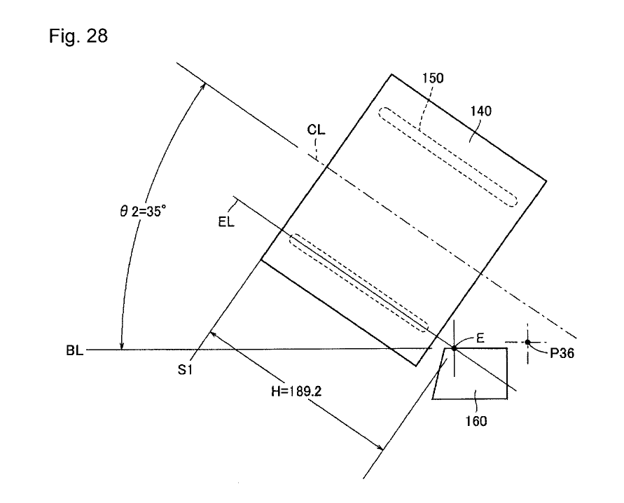

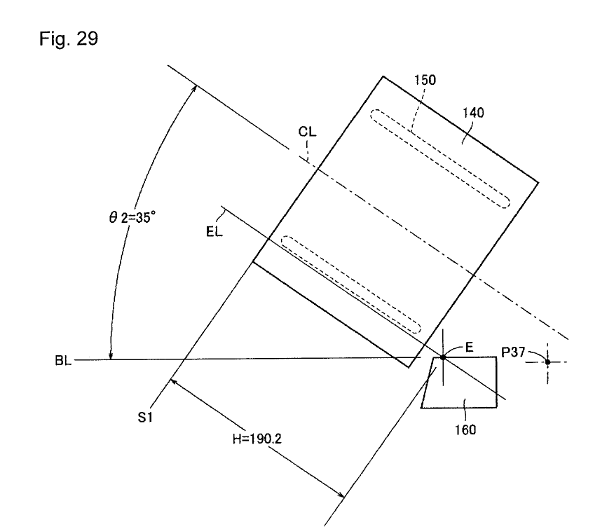

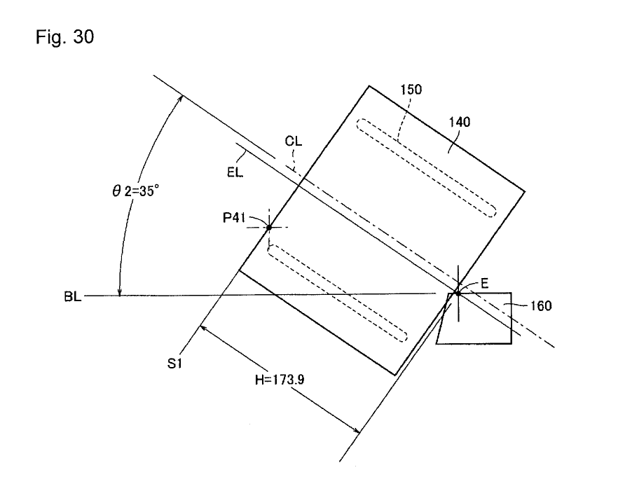

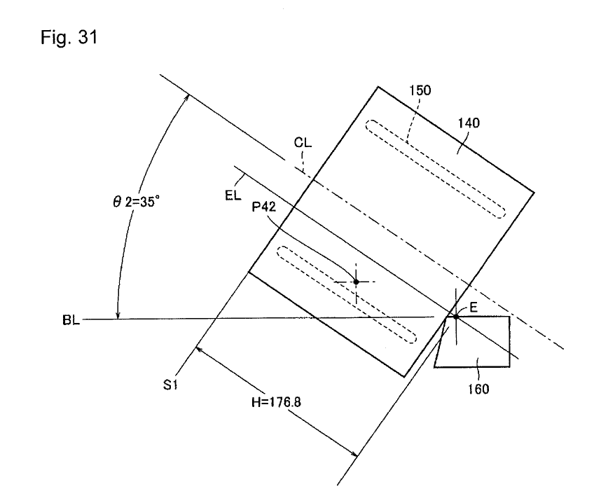

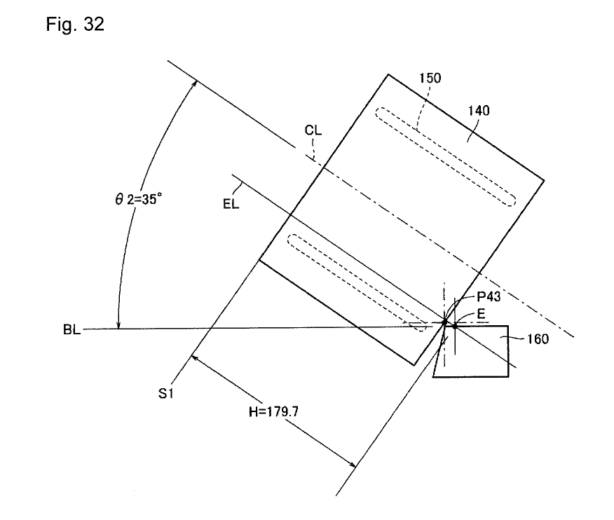

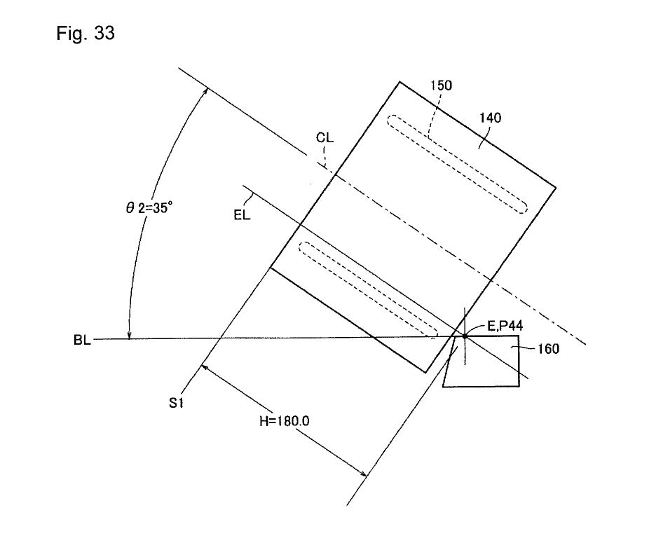

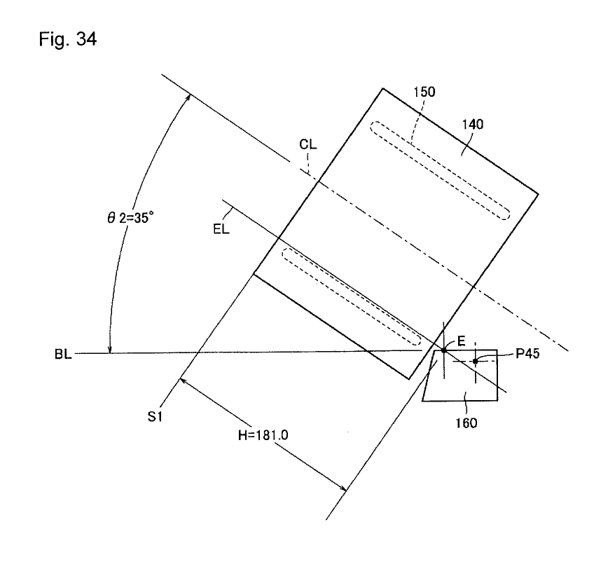

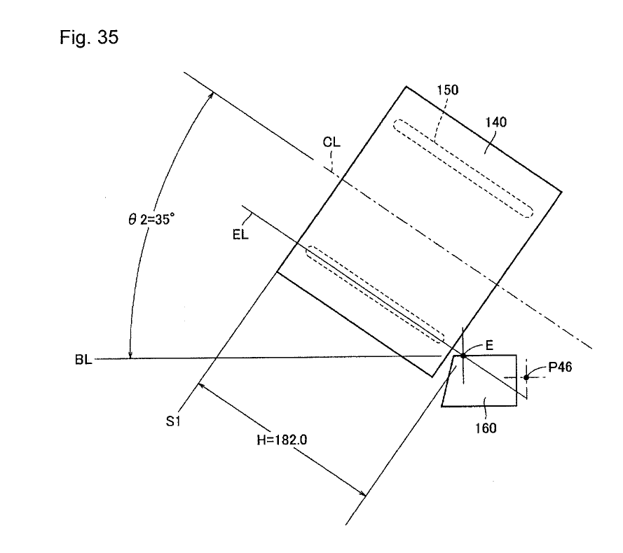

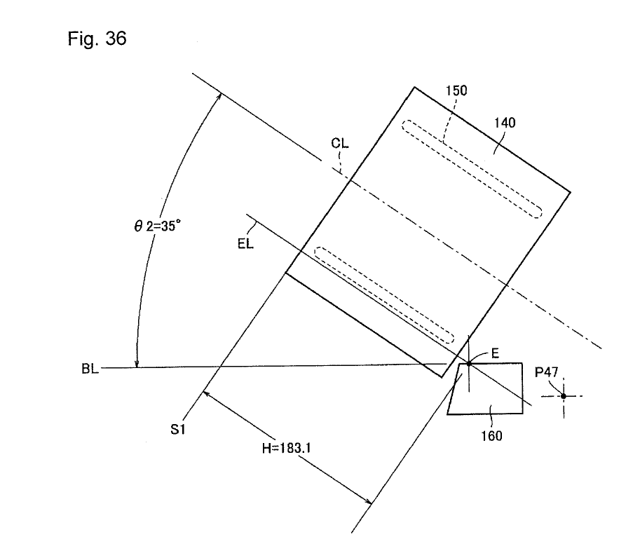

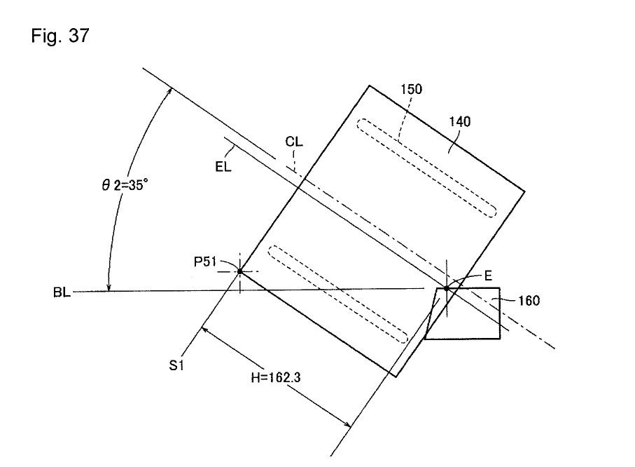

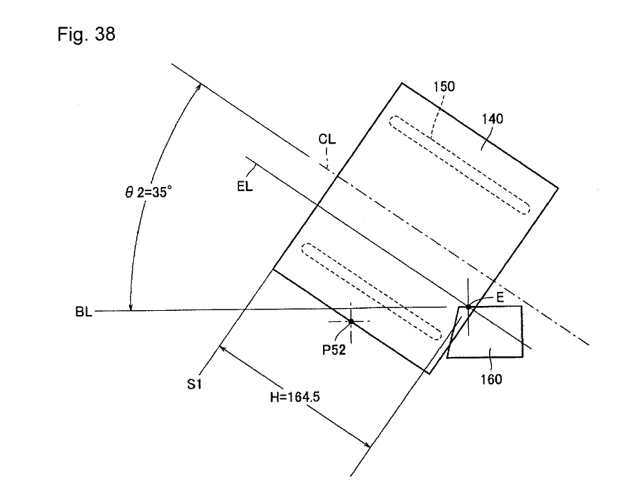

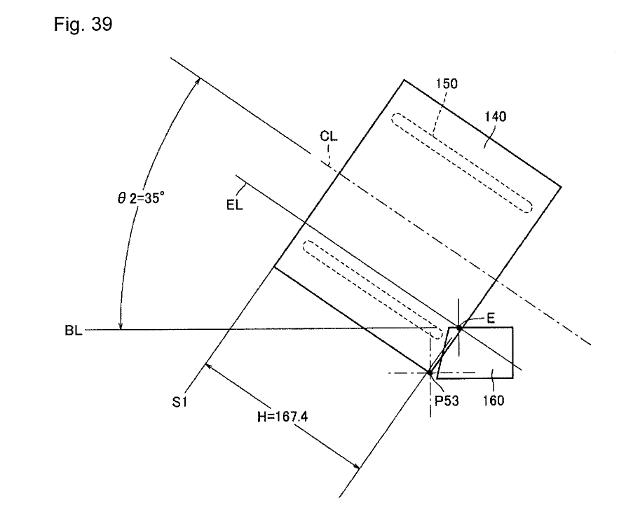

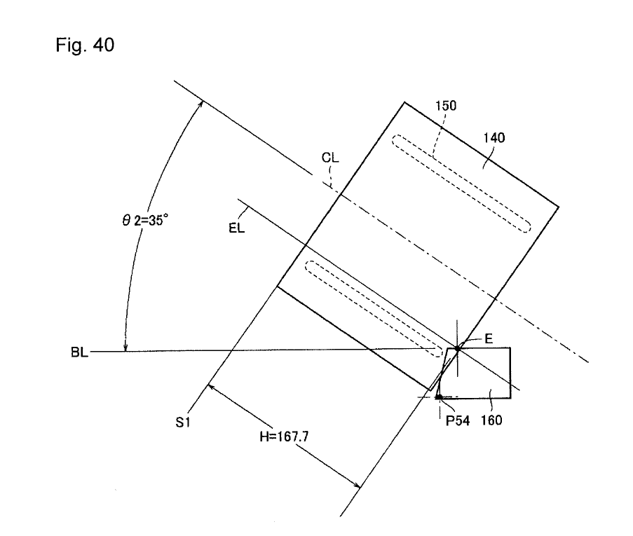

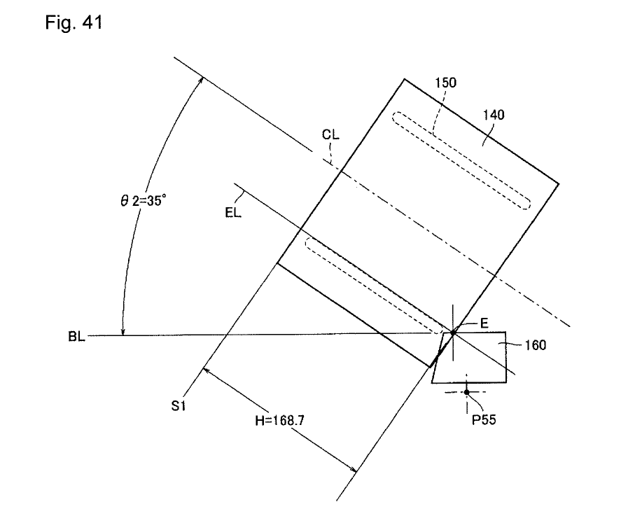

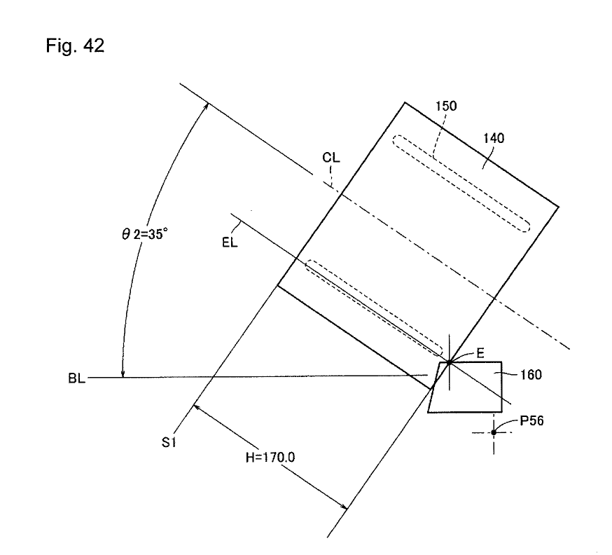

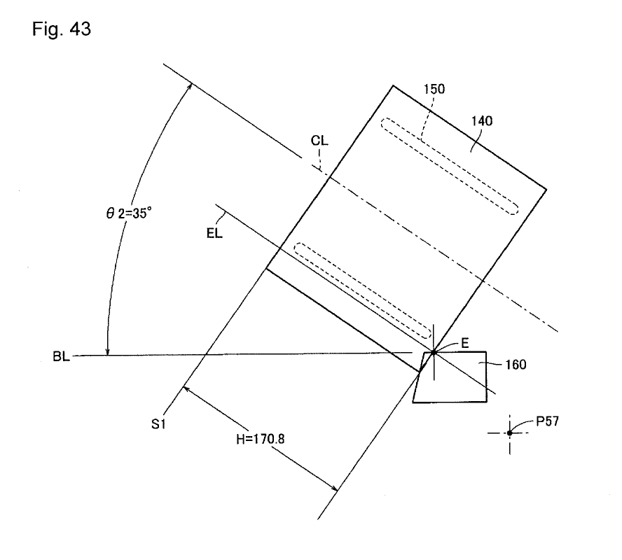

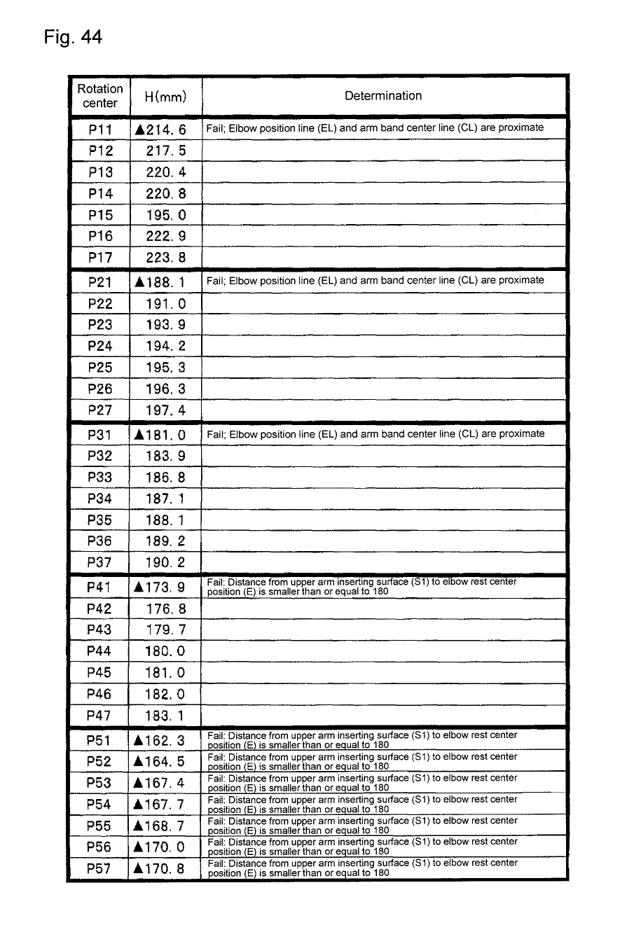

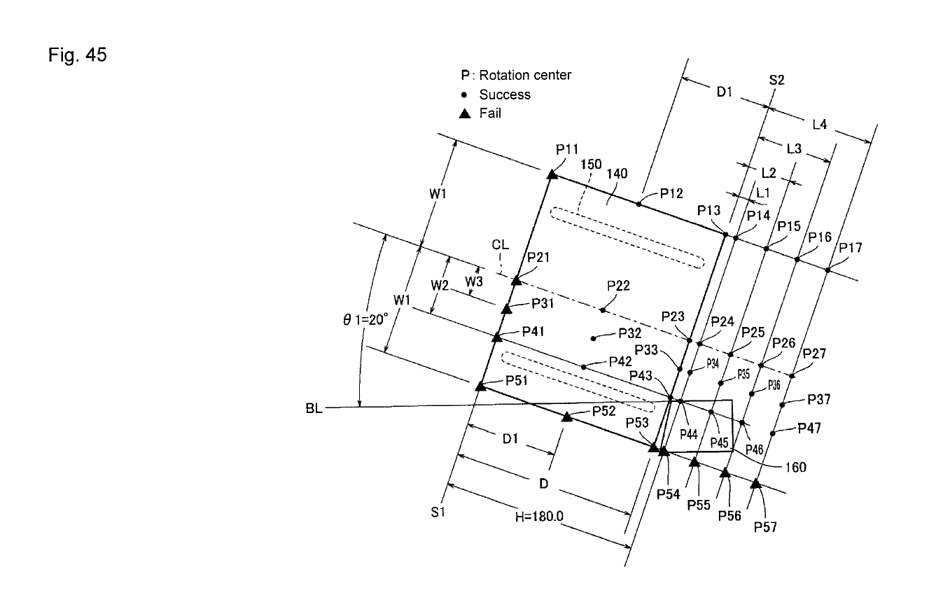

The position of the rotation center axis P will now be described with reference to FIG. 7 to FIG. 45. FIG. 7 is a schematic view showing a positional relationship of the living body inserting housing 140 and the elbow rest 160 when viewed from the axial direction of the rotation center axis, and FIG. 8 is a view showing the position of the rotation center axis. FIG. 9 to FIG. 43 are 1st to 35th views showing the rotation state of the living body inserting housing 140 at the position of the selected rotation center axis. FIG. 44 and FIG. 45 are first and second views showing the results obtained from the results of FIG. 9 to FIG. 43.

As shown in FIG. 7, according to one or more embodiments of the present invention, the living body inserting housing 140 has a rotation center axis enabling an angle formed by a plane BL including an elbow rest position center E at the elbow rest 160 and an arm band center axis CL of the arm band 150 to be movable in a range between a first angle (θ1) and a second angle (θ2) greater than the first angle (θ1). The arrow I in the figure indicates the upper arm inserting direction to the living body inserting housing 140.

As shown in FIG. 8, the distance H between the upper arm inserting surface S1 of the living body inserting housing 140 and the elbow rest position center E of the elbow rest 160 is set to 180.0 mm with the first angle (θ1) as 20 degrees. 35 areas P11 to P57 are shown for the position of the rotation center axis.

P11, P21, P31, P41, and P51 are arranged on the upper arm inserting surface S1 of the living body inserting housing 140 as the rotation center axes with the first angle (θ1) as 20 degrees. The dimension indicated with the symbol of the living body inserting housing 140 shown in FIG. 8 is as follows. W1=102.5 mm, W2=55 mm, W3=27.5 mm, D=170 mm, D1 (middle point S3 of D)=85 mm.

The dimension indicated with a symbol from the upper arm removing surface S2 of the living body inserting housing 140 is as follows. L1=10 mm, L2=40 mm, L3=70 mm, and L4=100 mm. The rotation center axes P12 to P17, P22 to P27, P32 to P37, P42 to P47, and P52 to P57 are arranged at the intersection of W1, W2, W3 and S2, S3, L1, L2, L3, L4.

A state in which the living body inserting housing 140 is rotated to the position of 35 degrees of the second angle (θ2) with each of rotation center axes P11 to P17, P21 to P27, P31 to P37, P41 to P47 and P51 to P57 set in the above manner as the rotation center axis is shown in FIG. 9 to FIG. 43. The distance H between the upper arm inserting surface S1 and the elbow rest position center E of the elbow rest 160 and the determination result read from the rotation state of the living body inserting housing 140 shown in FIG. 9 to FIG. 43 are shown in FIG. 44 and FIG. 45.

The blood pressure measurement of a short subject is assumed if the angle formed by the plane BL including the elbow rest position center E in the elbow rest 160 and the arm band center axis CL of the arm band 150 is the first angle (θ1) of 20 degrees, and the blood pressure measurement of a tall subject is assumed if the angle is the second angle (θ2) of 35 degrees.

Therefore, according to one or more embodiments of the present invention, because the distance H between the upper arm inserting surface S1 and the elbow rest position center E of the elbow rest 160 is set to 180 mm at the first angle (θ1), the distance H between the upper arm inserting surface S1 and the elbow rest position center E of the elbow rest 160 is greater than 180 mm when the second angle (θ2) is 35 degrees. The rotation center axis position at which the distance H becomes smaller than or equal to 180 mm is P41 to P43 and P51 to P57. The dimension of H at P42 and P43 is a dimension close to 180 mm or 176.8 mm and 179.7 mm, and thus, an accurate blood pressure measurement may be obtained for such two points.

When the elbow rest position line EL arranged parallel to the arm band center axis CL and passing through the elbow rest position center E is proximate to the arm band center axis CL, the insertion of the upper arm 220 to the living body inserting housing 140 becomes difficult. The rotation center axis position where the elbow rest position line EL and the arm band center axis CL are proximate is P11, P21, and P31.

The determination result shown in FIG. 44 is shown in FIG. 45. As shown in FIG. 45, if the rotation center axis P is positioned on the arm band center axis CL side rather than on the elbow rest position line EL when the rotation center axis is viewed from the axial direction, and the rotation center axis P is positioned on the elbow rest 160 side rather than on the axial center position S3 of the arm band 150 when viewed from the inserting direction of the upper arm 220 (direction of arrow I in FIG. 7) in the extending direction of the arm band center axis C, the distance from the upper arm inserting surface S1 of the living body inserting housing 140 to the elbow rest position center E at the second angle (θ2)=35 degrees is greater than the distance (H=180 mm) from the upper arm inserting surface S1 to the elbow rest position center E of the living body inserting housing 140 at the first angle (θ1)=20 degrees.

Therefore, in the blood pressure measurement device 100A according to one or more embodiments of the present invention, the rotation center axis is arranged at the position where the distance from the upper arm inserting surface S1 of the living body inserting housing 140 to the elbow rest position center E at the “second angle (θ2)=35 degrees”, which is an angle greater than the first angle, is greater than the distance from the upper arm inserting surface S1 to the elbow rest position center E of the living body inserting housing 140 at the “first angle (θ1)=20 degrees”.

When the rotation angle (θ) of the living body inserting housing 140 becomes small, the distance from the upper arm inserting surface S1 of the living body inserting housing to the position of the elbow rest becomes short, and hence, even a short subject can carry out the measurement comfortably in a natural position during the measurement.

In the embodiments described above, the upper arm type blood pressure measurement device for compressing the upper arm and measuring the blood pressure value has been illustrated, but embodiments of the present invention are not limited to such a blood pressure measurement device, and application can be made to the pulse wave detection device (pulse wave meter), and the like.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### Description of Reference Numerals

100A blood pressure measurement device

110 main body housing

110a base plate

114 operation unit

116 display unit

120 living body compressing air system

121 air pump

122 air valve

123 pressure sensor

124 air pump drive circuit

125 air valve drive circuit

126 amplifier

127 A/D converter

128 CPU

129 memory unit

140 living body inserting housing

140a supporting plate

150 arm band

152 living body compressing air bladder

154 air tube

160 elbow rest

170 arm rest

200 subject

201 elbow

210 arm

220 upper arm

230 chair

300 desk

BL plane

CL arm band center axis

EL elbow rest position line

E elbow rest position center

P, P11 to P57 rotation center axis

1. A blood pressure measurement device comprising:
a substantially cylindrical living body inserting housing in which an arm band with a hollowing opening, to which an upper arm of a subject is inserted from an axial direction, is arranged on an inner peripheral surface; and
a main body housing comprising an elbow rest for placing an elbow of the subject when the upper arm of the subject is passed through the living body inserting housing to take a measurement position,
wherein the living body inserting housing comprises a rotation center axis for an angle formed by a plane comprising an elbow rest position center in the elbow rest and an arm band center axis of the arm band to be movable in a range between a first angle and a second angle greater than the first angle, and
wherein when viewed from the axial direction, the rotation center axis is arranged at a position where a distance from an upper arm inserting surface of the living body inserting housing to the elbow rest position center in the second angle becomes greater than a distance from the upper arm inserting surface of the living body inserting housing to the elbow rest position center in the first angle.

2. The blood pressure measurement device according to claim 1, wherein when viewed from the axial direction, the rotation center axis is arranged parallel to the arm band center axis, and is positioned on the arm band center axis side rather than on an elbow rest position line passing through the elbow rest position center and positioned on the elbow rest side rather than on an axial center position of the arm band when viewed from an inserting direction of the upper arm in an extending direction of the arm band center axis.

3. The blood pressure measurement device according to claim 1, wherein the first angle is about 20 degrees, and the second angle is about 35 degrees.

4. The blood pressure measurement device according to claim 2, wherein the first angle is about 20 degrees, and the second angle is about 35 degrees.
